(19) United States
(12) Patent Application Publication
(10) Pub. No.: US 2006/0229261 A1
(43) Pub. Date: Oct. 12, 2006

(54) Acarbose methods and formulations for treating chronic constipation

(76) Inventor: John Devane, Co Roscommon (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/392,708

(22) Filed: Mar. 30, 2006

Related U.S. Application Data
(60) Provisional application No. 60/670,265, filed on Apr. 12, 2005.

(51) Int. Cl. A61K 31/70; A61K 31/155

(52) U.S. Cl. 514/25; 514/635

(57) ABSTRACT

The present invention is directed to a method for treating chronic constipation in a subject in need of such treatment comprising administering to the subject a dosage formulation comprising a therapeutically effective amount of acarbose, or a pharmaceutically acceptable salt thereof, and at least one pharmaceutically acceptable ingredient to control the release of the acarbose, wherein following administration, the dosage formulation releases the acarbose distal to the gastrointestinal sites at which acarbose is absorbed.

This application claims the benefit of priority to U.S. Provisional Patent Application No. 60/670,265, filed Apr. 12, 2005, the contents of which is incorporated herein by reference.

The present disclosure is directed to methods and formulations for treating chronic constipation. The methods and formulations include, but are not limited to, methods and formulations for delivering effective concentrations of acarbose. The methods and formulations further comprise at least one pharmaceutically acceptable ingredient to control the release of the acarbose, wherein following administration, the release of acarbose is distal to the gastrointestinal sites at which acarbose is absorbed. The present disclosure also relates to treating constipation as a symptom associated with other diseases and/or conditions such as irritable bowel syndrome (IBS).

Constipation occurs in up to 30% of the population. This symptom accounts for 1.2% of physician visits in the United States and is most frequently treated by primary care physicians. It is more common in females and increases with age. D. A. Drossman, The Functional Gastrointestinal Disorders and the Rome III Process, 45 Gut II1-II5 (Suppl. II 1999). There is also evidence to suggest that non-whites and persons of lower socioeconomic status are more likely to report chronic constipation. Almost a third of children with severe constipation will continue to suffer with symptoms beyond puberty.

Constipation comprises a group of functional disorders, which present as persistent, difficult, infrequent or seemingly incomplete defecation. Constipation has commonly been defined by three methods: 1) symptoms, in descending order of frequency, straining, hard stools, or scybala, unproductive calls (“want to but can't”), infrequent stools, incomplete evacuation; 2) parameters of defecation outside the 95^{th} percentile, e.g., less than three bowel movements per week, daily stool weight less than 35 g/day, or straining greater than 25% of the time; or 3) physiological measures such as prolonged whole gut transit or colonic transit as determined for instance by radio-opaque markers. D. A. Drossman, The Functional Gastrointestinal Disorders and the Rome III Process, 45 Gut II1-II5 (Suppl. II 1999).

As provided in Brooks Cash & William D. Chey, Update on the Management of Chronic Constipation: What Differentiates Chronic Constipation From IBS With Constipation, Medscape, at http://www.medscape.com/viewprogram/3375_pnt (Aug. 26, 2004), a variety of conditions and medications can be associated with chronic constipation, for example, primary or idiopathic constipation can be broadly divided into slow-transit constipation (i.e., colonic inertia) and dyssynergic defecation (i.e., anismus, outlet obstruction, pelvic floor dysfunction, pelvic floor dyssynergia, defecatory dysfunction). Physiologic abnormalities in patients with slow-transit constipation can include abnormal postprandial colonic motor function, autonomic dysfunction, and reduced numbers of colonic enterochromaffin cells and interstitial cells of Cajal. Dyssynergic defecation can occur as a consequence of the inability to coordinate actions of the abdominal musculature, anorectum, and pelvic floor musculature. An example is puborectalis dyssynergia, wherein the puborectalis sling fails to relax or paradoxically contracts with straining. This prevents straightening of the anorectal angle, which should precede the normal passage of stool. Structural abnormalities, such as a large rectocele, rectal intussusception, and obstructing sigmoidocele, can also contribute to constipation.

In addition, there can be significant overlap between patients with chronic constipation and irritable bowel syndrome-constipation (IBS-C) or constipation-dominant IBS. IBS can be characterized by abdominal discomfort or pain, bloating, and disturbed defecation. This disturbed defecation can take the form of constipation (IBS-C), diarrhea (IBS-D), or mixed/alternating bowel habits (IBS-M) with roughly equivalent distribution of the three subtypes.

Chronic constipation can also be a result of medications, endocrine disorders, and neurological disorders. For example, medications such as opiates, psychotropics, anticonvulsants, anticholinergics, dopaminergics, calcium channel blockers, bile acid binders, nonsterodial anti-inflammatory drugs, and supplements, i.e., calcium and iron, can initiate the onset of chronic constipation. Endocrine disorders such as diabetes mellitus, hypothyroidism, hyperparathyroidism, and pheochromocytoma similarly provoke the onset of chronic constipation. Moreover, chronic constipation can occur with both systemic (e.g., diabetic neuropathy, Parkinson's disease and Shy-Drager syndrome) and traumatic (e.g., spinal chord lesions) neurological disorders and. The term “constipation” as used herein, thus, encompasses conditions commonly identified as chronic constipation, functional constipation, chronic functional constipation, constipation, IBS-C, and/or other (non-chronic) constipation states.

Therapies for Chronic Constipation

The medical management of chronic constipation comprises lifestyle modifications in, e.g., diet and exercise, the use of bulking agents, e.g., psyllium, bran, methylcellulose, and calcium polycarbophil, and the administration of laxatives, including osmotic (e.g., polyethyleneglycol (PEG), lactulose, sorbitol, magnesium and phosphate salts), stimulants (e.g., senna-based and bisacodyl-based), and 5-hydroxytryptamine 4 (serotonin, 5-HT₄) receptor agonists (e.g., tegaserod).

Bulking Agents

Dietary fiber supplementation is believed to benefit constipated subjects by improving gastrointestinal transit and producing larger, softer stools. Dietary fiber supplementation can be, for example, achieved by increasing the ingestion of fiber-rich foods or by providing commercially available fiber supplements. Patients with chronic constipation can require greater doses of fiber than healthy volunteers to produce similar increases in stool volume and transit. Patients with severe colonic inertia or documented dyssynergic defecation can be less likely to improve with fiber.

Bulking agents can include psyllium, wheat bran, calcium polycarbophil, and methylcellulose. Three placebo-controlled trials of psyllium in patients with chronic constipation demonstrated improvements in stool frequency and consistency at doses ranging from 10 g/day to 24 g/day. L. J. Cheskin et al., Mechanisms of Constipation in Older Persons and Effects of Fiber Compared with Placebo, 43 J. American Geriatric Society 666-69 (1995); G. C. Fenn et al., A General Practice Study of the Efficacy of Regulanin Functional Constipation, 40 British J. Clinical Practice 192-97 (1986); and W. Ashraf et al., Effects of Psyllium Therapy on Stool Characteristics, Colon Transit and Anorectoal Function in Chronic Idiopathic Constipation, 9 Aliment Pharmacology & Therapeutics 639-47 (1995).

Despite the popularity of bran as a treatment for constipation, no randomized trials have shown improvements in stool frequency or consistency in patients with chronic constipation. There are no placebo-controlled trials examining calcium polycarbophil or methylcellulose in chronic constipated patients. In small trials comparing these agents versus psyllium, the data fail to demonstrate differences between agents in changes in stool frequency or consistency. R. Mamtani et al., A Calcium Salt of an Insoluble Synthetic Bulking Laxative in Elderlty Bedridden Nursing Home Residents, 8 J. American College Nutrition 554-56 (1989); and J. W. Hamilton et al., Clinical Evaluation of Methylcellulose as a Bulk Laxative, 33 Dig. Dis. Sci. 993-98 (1988).

Issues pertaining to convenience, palatability, and dose-dependent side effects (e.g., distention, bloating, and flatulence) limit patient compliance with instructions to use fiber supplements. Rare cases of anaphylaxis have been reported in patients taking psyllium.

Stool Softeners and Laxatives

Stool softeners can include, for example, dioctyl sodium sulfosuccinate and dioctyl calcium sulfosuccinate. Although these agents are commonly recommended for patients with constipation, there is little evidence to support their efficacy. Of four randomized controlled trials that evaluated stool softeners in patients with chronic constipation, only one, of three weeks' duration, found improvements in stool frequency compared with placebo. A. M. Fain et al., Treatment of Constipation in Geratric and Chronically Ill Patients: A Comparison, 71 South Med. J. 677-80 (1978). In another trial, psyllium was found to be superior to dioctyl sodium sulfosuccinate in improving stool frequency. J. W. McRorie et al., Psyllium is Superior to Docusate Sodium for Treatment of Chronic Constipation, 12 Aliment Pharmacology & Therapeutic 491-97 (1998).

Laxatives can be broadly divided into two categories: osmotic and stimulant laxatives. Examples of oral osmotic laxatives include poorly absorbed saccharides and saccharide derivatives, such as lactulose and sorbitol. These agents can increase stool volume and water content and, in so doing, stimulate peristalsis. Two trials have demonstrated that lactulose can be more effective than placebo at improving stool frequency and consistency. J. F. Sannders, Lactulose Syrup Assessed in a Double-Blind Study of Elderly Constipated Patients, 26 J. American Geriatric Society 236-39 (1978); A. Wesselius-De Casparis et al., Treatment of Chronic Constipation with Lactulose Syrup: Results of a Double-Blind Study, 9 Gut 84-86 (1968). Unfortunately, osmotic laxatives can sometimes be associated with the development of abdominal cramping and bloating.

Other examples of osmotic laxatives include incompletely absorbed salts comprising magnesium or sodium phosphate that produce a laxative effect by inducing a net flux of water into the bowel. Surprisingly, there are no randomized placebo-controlled trials assessing the efficacy of these agents in patients with chronic constipation. Hypermagnesemia and hyperphosphatemia can occur with these agents, such as in persons with renal disease or in the elderly.

Yet another example of an osmotic laxative is polyethylene glycol (PEG), which recently became available for the treatment of patients with occasional constipation. A number of randomized placebo-controlled trials in patients with constipation demonstrated significant improvements in stool frequency and consistency with PEG at doses of ranging from 17 g/day to 35 g/day. R. I. Andorsky and F. Goldner, Colonic Lavage Solution (Polyethylene Glycol Electrolyte Lavage Solution) as a Treatment for Chronic Constipation: A Double-Blind, Placebo-Controlled Study, 85 American J. Gastroenterol. 261-65 (1990); M. V. Cleveland et al., New Polyethylene Glycol Laxative for Treatment of Constipation in Adults: A Randomized, Double-Blind, Placebo-Controlled Study, 94 South Med. J. 478-81 (2001); E. Corazziari et al., Small Volume Isomotic Polyethylene Glycol Electrolyte Balanced Solution (PMF-100) in Treatment of Chronic Nonorganic Constipation, 41 Dig. Dis. Sci. 163642 (1996); and E. Corazziari et al., Long Term Efficacy, Safety, and Tolerability of Low Daily Doses of Isosmotic Polyethylene Glycol Electrolyte Balanced Solution (PMF-100) in the Treatment of Functional Chronic Constipation, 46 Gut 522-26 (2000). PEG, however, is not currently approved for use in treating chronic constipation.

Laxatives in the second category, stimulant laxatives, usually comprise bisacodyl, sodium picosulfate, or anthraquinone derivatives, such as cascara sagrada and senna. These agents have effects on bowel secretion and motility. There are no randomized placebo-controlled trials that assess the efficacy of stimulant laxatives in patients with chronic constipation. One comparative trial suggested that an “irritant laxative” was not as effective as lactulose in patients with constipation. P. Connolly et al., Comparison of “Duphalac” and “Irritant” Laxatives During and After Treatment of Chronic Constipation: A Preliminary Study, 2 Current Medical Research Opinions 620-25 (1974). Anthraquinone laxatives can induce melanosis coli, a reversible process that occurs as a consequence of colonic epithelial cell apoptosis and deposition of lipofuscin in macrophages.

Additional Treatments

Tegaserod, 3-(5-methoxy-1H-indol-3-ylmethylene)-N-pentylcarbazimidamide hydrogen maleate, is a 5-HT₄ (serotonin) agonist that stimulates the peristaltic reflex as well as chloride secretion and can affect visceral sensation. A number of, randomized, placebo-controlled trials indicate that tegaserod at a dose of 6 mg twice daily effectively improves global and individual symptoms in women patients with IBS-C. W. D. Chevy, Tegraserod and Other Sterotonergic Agents: What is the Evidence?, 3 Review Gastroenterol Disorders S35-S40 (2003); S. A. Muller-Lissner et al., Tegaserod, a 5-HT4 Receptor Partial Agonist, Relieves Symptoms of Irritable Bowel Syndrome in Patients with Abdominal Pain, Bloating and Constipation, 15 Aliment Pharmacology & Therapeutics 1655-66 (2001). Similar benefits, however, have not been demonstrated in male IBS patients.

In August 2004, the U.S. Food and Drug Administration approved a supplemental indication for tegaserod, allowing its use in the treatment of chronic idiopathic constipation in patients younger than 65 years. Tegaserod, however, must be used with caution including a specific precaution in relation to ischemic colitis.

In view of the foregoing, there remains a need in the art for pharmaceutical methods and formulations that can provide an effective, well tolerated treatment of constipation that avoids at least one of the many side effects and limitations associated with current therapies. The present invention solves at least one of the problems in the prior art and provides such methods and formulations for the treatment of constipation.

The present invention is directed to acarbose formulations for the treatment of constipation. Acarbose (PRECOSE®, Bayer Pharmaceuticals Corp.) is an oral alpha-glucosidase inhibitor traditionally used in the management of type 2 diabetes mellitus. See U.S. Pat. No. 4,904,769 directed to a purified acarbose composition and methods of producing the same, which is herein incorporated by reference. Derived by fermentation processes of a microorganism (Actinoplanes utahensis), acarbose has an empirical formula of C₂₅H₄₃NO₁₈. Current formulations of acarbose such as PRECOSE® are available in unit doses of 25 mg, 50 mg, and 100 mg tablets for oral use.

Compositions of acarbose for use as an antidiabetic agent are known. For example, in U.S. Pat. No. 5,965,163 describes solid dosage forms of pharmaceutically active substances, e.g., acarbose, in a matrix formed by a granulation process as an oral antidiabetic.

U.S. Patent Application Publication No. 2004/0096499 describes a solid dosage form comprising (i) a inner portion comprising an immediate-release formulation, where the low-dose active ingredient can be acarbose, and (ii) an outer portion comprising a modified-release formulation that provides a high dose, high solubility active ingredient. This combination uses agents with differing and complementary mechanisms of action to maximize therapeutic activity and reduce toxicity in the treatment of diabetes.

In addition, WO 00/28989 describes a composition combining a modified-release thiazolidnedione insulin sensitizer and another antidiabetic agent such as acarbose for treatment of diabetes. The goal is to provide a composition that allows once daily dosing while maintaining effective glycaemic control with no observed side effects.

Based on a recent investigation of elderly patients with diabetes mellitus, acarbose reduced the prolonged colonic transit time, a symptom prevalent in 60% diabetic neuropathy patients. Y. Ron et al., The Effect of Acarbose on the Colonic Transit Time of Elderly Long-Term Care Patients with Type 2 Diabetes Mellitus, 57 J Gerontol A Biol Sci Med Sci. M111-4 (2002). According to the data, acarbose could be used to treat the symptom of constipation found in this particular population, i.e., elderly diabetics, while controlling diabetes.

Acarbose is believed to delay the digestion of ingested carbohydrates resulting in a smaller influx of blood glucose following meals. Acarbose competitively and reversibly inhibits pancreatic alpha-amylase and membrane-bound intestinal alpha-glucoside hydrolase enzymes. By inhibiting pancreatic alpha-amylase, acarbose decreases complexes with starches and oligosaccharides in the lumen of the small intestine. By inhibiting membrane-bound intestinal alpha-glucoside hydrolase enzymes, acarbose decreases hydrolysis of oligosaccharides, trisaccharides and disaccharides to glucose and other monosaccharides in the brush border of the small intestine.

About 35% of an oral dose of acarbose is absorbed, primarily as inactive metabolites with about 2% absorbed as parent drug or active metabolite. Bacteria and enzymes in the the gastrointestinal tract are almost exclusively responsible for the metabolism of acarbose.

Given the absorption and metabolism characteristics of acarbose, the most common adverse reactions from the administration of PRECOSE® are gastrointestinal side effects. PRECOSE® Product Insert, Bayer Pharmaceuticals Corp. (08753825, R. 2, 2003). For example, reported gastrointestinal side effects include abdominal pain, diarrhea, and flatulence. Id.

Moreover, contraindications of acarbose, i.e., PRECOSE®, include a range of gastrointestinal conditions such as inflammatory bowel disease; colonic ulceration; arterial intestinal obstruction; chronic intestinal diseases associated with marked disorders of digestion or absorption in patients predisposed to intestinal obstruction; and conditions that can deteriorate as a result of increased gas formulation in the intestine. Id.

Acarbose is also used to treat obesity, for example, U.S. Pat. No. 6,849,609 describes a direct correlation between the administration of sustained-release acarbose and weight loss. According to this patent, the delivery of a sustained-release acarbose formulation to the small intestine produces a maximum inhibition of carbohydrate utilization, resulting in weight loss. As noted in U.S. Pat. No. 6,849,609, gastrointestinal symptoms associated with acarbose included flatulence, diarrhea, and abdominal pain. In addition, U.S. Pat. No. 5,643,874 describes pharmaceutical compositions for the treatment of obesity containing an effective amount of at least one but no more than two glucosidase and/or amylase inhibitors, a lipase inhibitor as an active substance, and pharmaceutical carriers for the treatment of obesity. The glucosidase and/or amylase inhibitor can be acarbose.

The present disclosure provides modified-release acarbose formulations and methods to treat chronic constipation and constipation as a symptom associated with diseases and/or conditions such as IBS. The modified-release formulations can be delayed-release and/or extended-release formulations.

For example, the present invention provides methods for treating constipation and/or treating constipation as a symptom associated with another disease and/or condition in a subject in need of such treatment. These methods include administering to the subject a dosage formulation comprising a therapeutically effective amount of acarbose, or a pharmaceutically acceptable salt thereof, and at least one pharmaceutically acceptable ingredient to control the release of the acarbose, wherein following administration, the dosage formulation releases the acarbose distal to the gastrointestinal sites at which acarbose is absorbed.

Constipation can be caused by conditions including, but not limited to, lifestyle habits, e.g., low dietary fiber and immobility, diseases of the peripheral and central nervous system, anatomic gastrointestinal obstructive lesions, endocrine disorders, metabolic disturbances, myotonic dystrophy, use of certain drugs, and/or can be a symptom of any of the foregoing conditions. Constipation can be treated with the administration of a delayed-release and/or extended-release formulation of acarbose, or a pharmaceutically acceptable salt thereof.

In all embodiments, the acarbose can comprise substantially pure acarbose, or a pharmaceutically acceptable salt thereof. The acarbose, or pharmaceutically acceptable salt thereof, can be administered in combination with at least one additional pharmaceutically active compound. In some embodiments, the at least one additional pharmaceutically active compound is capable of relieving constipation.

It is to be understood that both the foregoing general description and the following detailed description are exemplary and explanatory only and are not restrictive of the present invention, as claimed.

In order to further describe the present invention, the following terms and definitions are provided.

As used herein, the phrase “modified-release” formulation or dosage form includes a pharmaceutical preparation that achieves a desired release of the drug from the formulation. For example, a modified-release formulation can extend the influence or effect of a therapeutically effective dose of an active compound in a patient and as such, “modified-release” encompasses “extended-release” formulations. In addition to maintaining therapeutic levels of the active compound, a modified-release formulation can also be designed to delay the release of the active compound for a specified period and as such, “modified-release” also encompasses “delayed-release” formulations.

As used herein, the term “acarbose” means acarbose and any pharmaceutically acceptable salt thereof.

As used herein, the term “pharmaceutically acceptable ingredient” includes ingredients that are compatible with the other ingredients in a pharmaceutical formulation, such as the active ingredients, and not injurious to the patient when administered in acceptable amounts. Pharmaceutically acceptable ingredients that can be mentioned include, but are not limited to, for example, carriers, extenders, binders, disintegrating agents, solution-retarding agents, absorption accelerators, wetting agents, absorbents, lubricants, stabilizers, coloring agents, buffering agents, dispersing agents, preservatives, organic acids, water-soluble and water-insoluble polymers, enteric and non-enteric agents, and coatings.

As used herein, the term “pharmaceutically acceptable salt” includes salts that are physiologically tolerated by a patient. Such salts can be prepared from inorganic acids or bases and/or organic acids or bases. Examples of these acids and bases are well known to those of ordinary skill in the art. Such salts can be prepared from an inorganic and/or organic acid. Examples of suitable inorganic acids include, but are not limited to, hydrochloric, hydrobromic, hydroiodic, nitric, sulfuric, and phosphoric acid. Organic acids can be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like.

As used herein, the phrase “therapeutically effective amount” means the amount of acarbose (or pharmaceutically acceptable salt thereof), that alone and/or in combination with other drugs, provides a benefit in the prevention, treatment, and/or management of chronic constipation and constipation as a symptom associated with other diseases and/or conditions.

The present invention is directed to novel modified-release formulations that comprise acarbose, or a pharmaceutically acceptable salt thereof and methods of their use. Although not wishing to be bound by any particular theory, it is believed that the presence of acarbose reduces the incidence of chronic constipation and, further for example, constipation as a symptom associated with other diseases and/or conditions. In some embodiments, the modified-release formulation exhibits a release profile with delayed and/or extended-release properties.

The present invention is also directed to methods for treating chronic constipation comprising administering a delayed-release and/or extended-release formulation comprising an effective amount of acarbose or a pharmaceutically acceptable salt thereof, and at least one pharmaceutically acceptable ingredient to control the release of acarbose to a subject in need of such treatment. In some embodiments, the delayed-release and/or extended-release formulation releases acarbose distal to the gastrointestinal sites at which acarbose is absorbed.

Acarbose, as discussed above, is absorbed and metabolized in the gastrointestinal tract, e.g., the small intestine. As such, the delayed-release and/or extended-release formulations of the present invention are directed to, modifying the release of acarbose wherein the release of acarbose occurs distal to the gastrointestinal sites at which acarbose is absorbed. For example, the delayed-release formulation allows for maximum release at local non-absorption sites and can reduce release at sites capable of absorption, e.g., systemic absorption/exposure. By localizing the release of acarbose, the delayed-release formulations of the present invention can overcome at least one problem of conventional constipation therapies and provide for safer and more effective formulations.

The formulations and methods of the present invention are intended to include formulations and methods that are generic to treating constipation as a symptom associated with other diseases-and conditions.

The formulations of the present invention can exist as multi-unit or single-unit formulations. As used herein, “multi-unit” means a plurality of discrete or aggregated particles, beads, pellets, granules, tablets or mixtures thereof, for example, without regarding to their size, shape, or morphology. Single-unit formulations include, for example, tablets, caplets, and pills.

The methods and formulations of the present invention are intended to encompass all possible combinations of components that exhibit modified-release properties. For example, a formulation and/or method of the present invention can comprise components that exhibit extended-release and delayed-release properties. For example, a multipartiuclate formulation including both extended and delayed-release components can be combined in a capsule, which is then coated with to provide a delayed-release effect over a period of time ranging from 6 hours to 8 hours in duration.

In certain embodiments, the acarbose can be formulated into a liquid dosage form. Suitable formulations include emulsions, microemulsions, solutions, suspensions, syrups, and exlixirs. These formulations optionally include diluents commonly used in the art, such as, for example, water or solvents, solubilizing agents and emulsifiers, including, but not limited to, ethyl alcohol, isopropyl alcohol, ethyl carbonate, ethyl acetate, benzyl alcohol, benzyl benzoate, propylene glycol, 1,3-butylene glycol, oils, glycerol, tetrahydrofuryl alcohol, polyethylene glycols, fatty acid esters of sorbitan, and mixtures thereof. In addition, the liquid formulations optionally include adjuvants such as wetting agents, emulsifying and suspending agents, sweetening, flavoring, coloring, perfuming, and preservative agents. Suitable suspension agents include, but are not limited to, ethoxylated isostearyl alcohols, polyoxyethylene sorbitol and sorbitan esters, microcrystalline cellulose, aluminum metahydroxide, bentonite, agar-agar and tragacanth, and mixtures thereof. The liquid formulations can be delivered as-is, or can be provided in hard or soft capsules.

The amount of suspending agent present will vary according to the particular suspending agent used, and the presence or absence of other ingredients that have an ability to act as a suspending agent or contribute significantly to the viscosity of the formulation. The suspension can also comprise ingredients that improve its taste, for example, sweeteners; bitter-taste maskers, such as sodium chloride; taste-masking flavors, such as contramarum; flavor enhancers, such as monosodium glutamate; and flavoring agents. Examples of sweeteners include bulk sweeteners, such as sucrose, hydrogenated glucose syrup, the sugar alcohols sorbitol and xylitol; and sweetening agents such as sodium cyclamate, sodium saccharin, aspartame, and ammonium glycyrrhizinate. The liquid formulations can further comprise at least one buffering agent, as needed, to maintain a desired pH.

Soft Gelatin Capsules

The formulations of the present invention can also be prepared as liquids, which can be filled into soft gelatin capsules. For example, the liquid can include a solution, suspension, emulsion, microemulsion, precipitate, or any other desired liquid media carrying the acarbose. The liquid can be designed to improve the solubility of the acarbose upon release, or can be designed to form a drug-comprising emulsion or dispersed phase upon release. Examples of such techniques are well known in the art. Soft gelatin can be coated, as desired, with a functional coating to delay the release of the drug.

The compositions of the present invention can also be formulated into other dosage forms that modify the release of the active agent, i.e., acarbose, or a pharmaceutically acceptable salt thereof. Examples of suitable modified-release formulations that can be used in accordance with the present invention include, but are not limited to, matrix systems, osmotic pumps, and membrane-controlled dosage forms. Each of these types of dosage forms are briefly described below. A more detailed discussion of such forms can also be found in, for example, The Handbook of Pharmaceutical Controlled Release Technology, D. L. Wise (ed.), Marcel Dekker, Inc., New York (2000); and also in Treatise on Controlled Drug Delivery: Fundamentals, Optimization, and Applications, A. Kydonieus (ed.), Marcel Dekker, Inc., New York, (1992), the relevant contents of each of which are hereby incorporated by reference for this purpose.

Matrix-Based Dosage Forms

In some embodiments, the modified-release and/or delayed-release formulations of the present invention are provide as matrix-based dosage forms. Matrix formulations according to the invention can include hydrophilic, e.g., water-soluble, and/or hydrophobic, e.g., water-insoluble, polymers. The matrix formulations of the present invention can be prepared with functional coatings, which can be enteric, e.g., exhibiting a pH-dependent solubility, or non-enteric, e.g., exhibiting a pH-independent solubility.

Matrix formulations of the present invention can be prepared by using, for example, direct compression or wet granulation. A functional coating, as noted above, can then be applied in accordance with the invention. Additionally, a barrier or sealant coat can be applied over a matrix tablet core before application of a functional coating. The barrier or sealant coat can serve the purpose of separating an active ingredient from a functional coating, which can interact with the active ingredient, or it can prevent moisture from contacting the active ingredient. Details of barriers and sealants are provided below.

In a matrix-based dosage form in accordance with the present invention, the acarbose and the at least one pharmaceutically acceptable ingredient can be dispersed within a polymeric matrix, which typically comprises at least one water-soluble polymer and at least one water-insoluble polymer. The drug can be released from the dosage form by diffusion and/or erosion. Such matrix systems are described in detail by Wise and Kydonieus, supra.

Suitable water-soluble polymers include, but are not limited to, polyvinyl alcohol, polyvinylpyrrolidone, methylcellulose, hydroxypropylcellulose, hydroxypropylmethyl cellulose, or polyethylene glycol, and/or mixtures thereof.

Suitable water-insoluble polymers include, but are not limited to, ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(ethylene oxide), poly(ethylene terephthalate), poly(vinyl isobutyl ether), poly(vinyl acetate), poly(vinyl chloride), or polyurethane, and/or mixtures thereof.

Suitable pharmaceutically acceptable excipients include, but are not limited to, carriers, such as sodium citrate and dicalcium phosphate; fillers or extenders, such as stearates, silicas, gypsum, starches, lactose, sucrose, glucose, mannitol, talc, and silicic acid; binders, such as hydroxypropyl methylcellulose, hydroxymethylcellulose, alginates, gelatin, polyvinylpyrrolidone, sucrose, and acacia; humectants, such as glycerol; disintegrating agents, such as agar, calcium carbonate, potato and tapioca starch, alginic acid, certain silicates, EXPLOTAB™, crospovidone, and sodium carbonate; solution-retarding agents, such as paraffin; absorption accelerators, such as quaternary ammonium compounds; wetting agents, such as cetyl alcohol and glycerol monostearate; absorbents, such as kaolin and bentonite clay; lubricants, such as talc, calcium stearate, magnesium stearate, solid polyethylene glycols, and sodium lauryl sulfate; stabilizers, such as fumaric acid; coloring agents; buffering agents; dispersing agents; preservatives; organic acids; and organic bases. The aforementioned excipients are given as examples only and are not meant to include all possible choices. Additionally, many excipients can have more than one role or function, or be classified in more than one group; the classifications are descriptive only, and not intended to limit any use of an exemplified excipient.

The aforementioned excipients are given as examples only and are not meant to include all possible choices. Solid formulations can also be prepared as fillers in soft and hard-filled gelatin capsules using excipients such as lactose or milk sugars, high molecular weight polyethylene glycols, and the like. Any of these dosage forms can optionally be scored or prepared with coatings and shells, such as enteric coatings and coatings for modifying the rate of release, examples of which are well known in the pharmaceutical-formulating art.

In some embodiments, a matrix-based dosage form comprises acarbose; a filler, such as starch, lactose, or microcrystalline cellulose (AVICEL™); a binder/controlled-release polymer, such as hydroxypropyl methylcellulose or polyvinyl pyrrolidone; a lubricant, such as magnesium stearate or stearic acid; a surfactant, such as sodium lauryl sulfate or polysorbates; and a glidant, such as colloidal silicon dioxide (AEROSIL™) or talc. In some embodiments, a disintegrant such as EXPLOTAB™, crospovidone, or starch is also included.

The amounts and types of polymer(s), and the ratio of water-soluble polymer(s) to water-insoluble polymer(s) in the presently disclosed formulations are generally selected to achieve a desired release profile of acarbose, as described below. For example, by increasing the amount of water-insoluble polymer relative to the amount of water-soluble polymer, the release of the drug can be delayed or slowed. This is due, in part, to an increased impermeability of the polymeric matrix, and, in some cases, to a decreased rate of erosion during transit through the gastrointestinal tract.

Osmotic Pump Dosage Forms

In various embodiments, the modified-release formulations of the present invention are provided as osmotic pump dosage forms. In an osmotic pump dosage form, a core comprising the acarbose and optionally at least one osmotic excipient can be encased by a selectively permeable membrane having at least one orifice. The selectively permeable membrane is generally permeable to water, but impermeable to the drug. When body fluids contact the system, water penetrates through the selectively permeable membrane into the core containing the drug and optional osmotic excipients. The osmotic pressure increases within the dosage form, and the drug is released through the at least one orifice in an attempt to equalize the osmotic pressure across the selectively permeable membrane.

In more complex pumps, the dosage form can comprise at least two internal compartments in the core. The first compartment comprises the drug and the second compartment can comprise at least one polymer, which swells on contact with aqueous fluid. After ingestion, this polymer swells into the drug-comprising compartment, diminishing the volume occupied by the drug, thereby enabling one to optimize the delivery of the drug from the device at a controlled rate over a modified period or delivery based on the pH of the particular environment.

Osmotic pumps are well known in the art. For example, U.S. Pat. Nos. 4,088,864, 4,200,098, and 5,573,776, each of which is hereby incorporated by reference for this purpose, describe osmotic pumps and methods of their manufacture. The osmotic pumps useful in accordance with the present invention can be formed by compressing a tablet of an osmotically active drug, or an osmotically inactive drug in combination with an osmotically active agent, and then coating the tablet with a selectively permeable membrane that is permeable to an exterior aqueous-based fluid but impermeable to the drug and/or osmotic agent.

At least one delivery orifice can be drilled through the selectively permeable membrane wall. Alternatively, the at least one orifice in the wall can be formed by incorporating leachable pore-forming materials in the wall. In operation, the exterior aqueous-based fluid is imbibed through the selectively permeable membrane wall and contacts the drug to form a solution or suspension of the drug. The drug solution or suspension is then pumped out through the orifice as fresh fluid is imbibed through the selectively permeable membrane. This enables one to optimize the delivery of the drug from the device at a modified rate over an extended period or delivery based on the pH of the particular environment.

Typical materials for the selectively permeable membrane include selectively permeable polymers known in the art to be useful in osmosis and reverse osmosis membranes, such as cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloracetate, cellulose dipalmitate, cellulose dioctanoate, cellulose dicaprylate, cellulose dipentanate, cellulose acetate valerate, cellulose acetate succinate, cellulose propionate succinate, methyl cellulose, cellulose acetate p-toluene sulfonate, cellulose acetate butyrate, lightly cross-linked polystyrene derivatives, cross-linked poly(sodium styrene sulfonate), poly(vinylbenzyltrimethyl ammonium chloride), and/or mixtures thereof.

The osmotic agents that can be used in the pump are typically soluble in the fluid that enters the device following administration, resulting in an osmotic pressure gradient across the selectively permeable wall against the exterior fluid. Suitable osmotic agents include, but are not limited to, magnesium sulfate, calcium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, glucose, hydrophilic polymers such as cellulose polymers, and/or mixtures thereof.

As discussed above, the osmotic pump dosage form can comprise a second compartment comprising a swellable polymer. Suitable swellable polymers typically interact with water and/or aqueous biological fluids, which causes them to swell or expand to an equilibrium state. Acceptable polymers exhibit the ability to swell in water and/or aqueous biological fluids, retaining a significant portion of such imbibed fluids within their polymeric structure, so as to increase the hydrostatic pressure within the dosage form. The polymers can swell or expand to a very high degree, usually exhibiting a 2- to 50-fold volume increase. The polymers can be non-cross-linked or cross-linked. In some embodiments, the swellable polymers are hydrophilic polymers. Suitable polymers include, but are not limited to, poly(hydroxy alkyl methacrylate) having a molecular weight of from about 30,000 to about 5,000,000; kappa-carrageenan; polyvinylpyrrolidone having a molecular weight of from about 10,000 to about 360,000; anionic and cationic hydrogels; polyelectrolyte complexes; poly(vinyl alcohol) having low amounts of acetate, cross-linked with glyoxal, formaldehyde, or glutaraldehyde, and having a degree of polymerization from about 200 to about 30,000; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene, or isobutylene; water-swellable polymers of N-vinyl lactams; and/or mixtures of any of the foregoing.

The term “orifice” as used herein includes means and methods suitable for releasing the drug from the dosage form. The expression includes at least one aperture or orifice that has been bored through the selectively permeable membrane by mechanical procedures. Alternatively, an orifice can be formed by incorporating an erodible element, such as a gelatin plug, in the selectively permeable membrane. In such cases, the pores of the selectively permeable membrane form a “passageway” for the passage of the drug. Such “passageway” formulations are described, for example, in U.S. Pat. Nos. 3,845,770 and 3,916,899, the relevant disclosures of which are incorporated herein by reference for this purpose.

The osmotic pumps useful in accordance with this invention can be manufactured by techniques known in the art. For example, the drug and other ingredients can be milled together and pressed into a solid having the desired dimensions (e.g., corresponding to the first compartment). The swellable polymer is then formed, placed in contact with the drug, and both are surrounded with the selectively permeable agent. If desired, the drug component and polymer component can be pressed together before applying the selectively permeable membrane. The selectively permeable membrane can be applied by any suitable method, for example, by molding, spraying, or dipping.

Membrane-Controlled Dosage Forms

The modified-release formulations of the present invention can also be provided as membrane-controlled formulations. Membrane-controlled formulations of the present disclosure can be made by preparing a rapid release core, which can be a monolithic (e.g., tablet) or multi-unit (e.g., pellet) type, and coating the core with a membrane. The membrane-controlled core can then be further coated with a functional coating. In between the membrane-controlled core and the functional coating, a barrier or sealant can be applied. The barrier or sealant can alternatively, or additionally, be provided between the rapid release core and the membrane coating. Details of membrane-controlled dosage forms are provided below.

In certain embodiments, the acarbose is provided in a multiparticulate membrane-controlled formulation. Acarbose can be formed into an active core by applying the drug to a nonpareil seed having an average diameter in the range of about 0.4 to about 1.1 mm or about 0.85 to about 1.00 mm. The acarbose can be applied with or without additional excipients onto the inert cores, and can be sprayed from solution or suspension using a fluidized-bed coater (e.g., Wurster coating) or pan coating system. Alternatively, the acarbose can be applied as a powder onto the inert cores using a binder to bind the acarbose onto the cores. Active cores can also be formed by extrusion of the core with suitable plasticizers (described below) and any other processing aids as necessary.

The delayed-release and/or extended-release formulations of the present invention comprise at least one polymeric material, which is applied as a membrane coating to the drug-containing cores. Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water-insoluble polymers include, but are not limited to, ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(ethylene oxide), poly(ethylene terephthalate), poly(vinyl isobutyl ether), poly(vinyl acetate), poly(vinyl chloride), or polyurethane, and/or mixtures thereof.

EUDRAGIT® polymers (available from Rohm Pharma) are polymeric lacquer substances based on acrylates and/or methacrylates. A suitable polymer that is freely permeable to the active ingredient and water is EUDRAGIT® RL. A suitable polymer that is slightly permeable to the active ingredient and water is EUDRAGIT® RS. Other suitable polymers that are slightly permeable to the active ingredient and water, and exhibit a pH-dependent permeability include, but are not limited to, EUDRAGIT® L, EUDRAGIT® S, and EUDRAGIT® E.

EUDRAGIT® RL and RS are acrylic resins comprising copolymers of acrylic and methacrylic acid esters with a low content of quaternary ammonium groups. The ammonium groups are present as salts and give rise to the permeability of the lacquer films. EUDRAGIT® RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT® L is an anionic polymer synthesized from methacrylic acid and methacrylic acid methyl ester. It is insoluble in acids and pure water. It becomes soluble in neutral to weakly alkaline conditions. The permeability of EUDRAGIT® L is pH dependent. Above pH 5.0, the polymer becomes increasingly permeable.

In various embodiments comprising a membrane-controlled dosage form, the polymeric material comprises methacrylic acid co-polymers, ammonio methacrylate co-polymers, or mixtures thereof. Methacrylic acid co-polymers such as EUDRAGIT® S and EUDRAGIT® L (Rohm Pharma) are suitable for use in the controlled release formulations of the present invention. These polymers are gastroresistant and enterosoluble polymers. Their polymer films are insoluble in pure water and diluted acids. They dissolve at higher pHs, depending on their content of carboxylic acid. EUDRAGIT® S and EUDRAGIT® L can be used as single components in the polymer coating or in combination in any ratio. By using a combination of the polymers, the polymeric material can exhibit a solubility at a pH between the pHs at which EUDRAGIT® L and EUDRAGIT® S are separately soluble.

The membrane coating can comprise a polymeric material comprising a major proportion (i.e., greater than 50% of the total polymeric content) of at least one pharmaceutically acceptable water-soluble polymers, and optionally a minor proportion (i.e., less than 50% of the total polymeric content) of at least one pharmaceutically acceptable water insoluble polymers. Alternatively, the membrane coating can comprise a polymeric material comprising a major proportion (i.e., greater than 50% of the total polymeric content) of at least one pharmaceutically acceptable water insoluble polymers, and optionally a minor proportion (i.e., less than 50% of the total polymeric content) of at least one pharmaceutically acceptable water-soluble polymer.

Ammonio methacrylate co-polymers such as EUDRAGIT® RS and EUDRAGIT® RL (Rohm Pharma) are suitable for use in the modified release formulations of the present invention. These polymers are insoluble in pure water, dilute acids, buffer solutions, or digestive fluids over the entire physiological pH range. The polymers swell in water and digestive fluids independently of pH. In the swollen state, they are then permeable to water and dissolved active agents. The permeability of the polymers depends on the ratio of ethylacrylate (EA), methyl methacrylate (MMA), and trimethylammonioethyl methacrylate chloride (TAMCl) groups in the polymer. Those polymers having EA:MMA:TAMCl ratios of 1:2:0.2 (EUDRAGIT® RL) are more permeable than those with ratios of 1:2:0.1 (EUDRAGIT® RS). Polymers of EUDRAGIT® RL are insoluble polymers of high permeability. Polymers of EUDRAGIT® RS are insoluble films of low permeability.

The amino methacrylate co-polymers can be combined in any desired ratio, and the ratio can be modified to modify the rate of drug release. For example, a ratio of EUDRAGIT® RS: EUDRAGIT® RL of 90:10 can be used. Alternatively, the ratio of EUDRAGIT® RS: EUDRAGIT® RL can be about 100:0 to about 80:20, or about 100:0 to about 90:10, or any ratio in between. In such formulations, the less permeable polymer EUDRAGIT® RS would generally comprise the majority of the polymeric material.

The amino methacrylate co-polymers can be combined with the methacrylic acid co-polymers within the polymeric material in order to achieve the desired delay in the release of the drug. Ratios of ammonio methacrylate co-polymer (e.g., EUDRAGIT® RS) to methacrylic acid co-polymer in the range of about 99:1 to about 20:80 can be used. The two types of polymers can also be combined into the same polymeric material, or provided as separate coats that are applied to the core.

In addition to the EUDRAGIT® polymers described above, a number of other such copolymers can be used to control drug release. These include methacrylate ester co-polymers (e.g., EUDRAGIT® NE 30D). Further information on the EUDRAGIT® polymers can be found in “Chemistry and Application Properties of Polymethacrylate Coating Systems,” in Aqueous Polymeric Coatings for Pharmaceutical Dosage Forms, ed. James McGinity, Marcel Dekker Inc., New York, pg 109-114.

In addition to the EUDRAGIT® polymers discussed above, other enteric, or pH-dependent, polymers can be used. Such polymers can include phthalate, butyrate, succinate, and/or mellitate groups. Such polymers include, but are not limited to, cellulose acetate phthalate, cellulose acetate succinate, cellulose hydrogen phthalate, cellulose acetate trimellitate, hydroxypropyl-methylcellulose phthalate, hydroxypropylmethylcellulose acetate succinate, starch acetate phthalate, amylose acetate phthalate, polyvinyl acetate phthalate, and polyvinyl butyrate phthalate.

The coating membrane can further comprise at least one soluble excipient to increase the permeability of the polymeric material. Suitably, the at least one soluble excipient is selected from among a soluble polymer, a surfactant, an alkali metal salt, an organic acid, a sugar, and a sugar alcohol. Such soluble excipients include, but are not limited to, polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid, sugars such as dextrose, fructose, glucose, lactose, and sucrose, sugar alcohols such as lactitol, maltitol, mannitol, sorbitol, and xylitol, xanthan gum, dextrins, and maltodextrins. In some embodiments, polyvinyl pyrrolidone, mannitol, and/or polyethylene glycol can be used as soluble excipients. The at least one soluble excipient can be used in an amount ranging from about 1% to about 10% by weight, based on the total dry weight of the polymer. The coating process can be carried out by any suitable means, for example, by using a perforated pan system such as the GLATT™, ACCELACOTA™, and/or HICOATER™ apparatuses.

In certain embodiments, the polymeric material comprises at least one water-insoluble polymer, which are also insoluble in gastrointestinal fluids, and at least one water-soluble pore-forming compound. For example, the water-insoluble polymer can comprise a terpolymer of polyvinylchloride, polyvinylacetate, and/or polyvinylalcohol. Suitable water-soluble pore-forming compounds include, but are not limited to, saccharose, sodium chloride, potassium chloride, polyvinylpyrrolidone, and/or polyethyleneglycol. The pore-forming compounds can be uniformly or randomly distributed throughout the water insoluble polymer. Typically, the pore-forming compounds comprise about 1 part to about 35 parts for each about 1 to about 10 parts of the water insoluble polymers.

When such dosage forms come in to contact with the dissolution media (e.g., intestinal fluids), the pore-forming compounds within the polymeric material dissolve to produce a porous structure through which the drug diffuses. Such formulations are described in more detail in U.S. Pat. No. 4,557,925, which relevant part is incorporated herein by reference for this purpose. The porous membrane can also be coated with an enteric coating, as described herein, to inhibit release in the stomach.

In some embodiments, such pore-forming modified-release dosage forms comprise acarbose; a filler, such as starch, lactose, or microcrystalline cellulose (AVICEL™); a binder/controlled release polymer, such as hydroxypropyl methylcellulose or polyvinyl pyrrolidone; a disintegrant, such as, EXPLOTAB™, crospovidone, or starch; a lubricant, such as magnesium stearate or stearic acid; a surfactant, such as sodium lauryl sulfate or polysorbates; and a glidant, such as colloidal silicon dioxide (AEROSIL™) or talc.

The polymeric material can also include at least one auxiliary agent such as fillers, plasticizers, and/or anti-foaming agents. Representative fillers include talc, fumed silica, glyceryl monostearate, magnesium stearate, calcium stearate, kaolin, colloidal silica, gypsum, micronized silica, and magnesium trisilicate. The quantity of filler used typically ranges from about 2% to about 300% by weight, and can range from about 20% to about 100%, based on the total dry weight of the polymer. In some embodiments, talc is the filler.

The coating membranes and functional coatings as well, can also include a material that improves the processing of the polymers. Such materials are generally referred to as plasticizers and include, for example, adipates, azelates, benzoates, citrates, isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In various embodiments, the plasticizer is dibutyl sebacate. The amount of plasticizer used in the polymeric material can range from about 10% to about 50%, for example, about 10%, 20%, 30%, 40%, or 50%, based on the weight of the dry polymer.

Anti-foaming agents can also be included. In some embodiments, the anti-foaming agent is simethicone. The amount of anti-foaming agent used can comprise from about 0% to about 0.5% of the final formulation.

The amount of polymer to be used in the membrane-controlled formulations is typically adjusted to achieve the desired drug delivery properties, including the amount of drug to be delivered, the rate and location of drug delivery, the time delay of drug release, and the size of the multiparticulates in the formulation. The amount of polymer applied typically provides an about 10% to about 100% weight gain to the cores. In some embodiments, the weight gain from the polymeric material ranges from about 25% to about 70%.

A polymeric membrane can include components in addition to polymers, such as, for example, fillers, plasticizers, stabilizers, or other excipients and processing aids. One example of an additional component of the membrane is sodium hydrogen carbonate, which can act as a stabilizer.

The combination of all solid components of the polymeric material, including co-polymers, fillers, plasticizers, and optional excipients and processing aids, can provide an about 10% to about 450% weight gain on the cores. In various embodiments, the weight gain is about 30% to about 160%.

The polymeric material can be applied by any known method, for example, by spraying using a fluidized bed coater (e.g., Wurster coating) or pan coating system. Coated cores are typically dried or cured after application of the polymeric material. Curing means that the multiparticulates are held at a controlled temperature for a time sufficient to provide stable release rates. Curing can be performed, for example, in an oven or in a fluid bed drier. Curing can be carried out at any temperature above room temperature, which can be above the glass transition temperature of the relevant polymer.

A sealant or barrier can also be applied to the polymeric coating. Alternatively, or additionally, a sealant or barrier layer can be applied to the core prior to applying the polymeric material. A sealant or barrier layer is generally not intended to modify the release of acarbose, but might, depending on how it is formulated. Suitable sealants or barriers are permeable or soluble agents such as hydroxypropyl methylcellulose, hydroxypropyl cellulose, hydroxypropyl ethylcellulose, polyvinyl pyrrolidone, and xanthan gum. An outer sealant/barrier, for example, can be used to improve moisture resistance of the entire formulation. A sealant/barrier between the core and the coating, for example, can be used to protect the core contents from an outer polymeric coating that can exhibit pH-dependent or pH-independent dissolution properties. Additionally, there can be instances in which both effects are desired, i.e., moisture resistance and core protection, in which a sealant/barrier is applied between the core and the polymeric membrane coating, and then outside the polymeric membrane coating.

Other agents can be added to improve the processability of a sealant or barrier layer. Such agents include talc, colloidal silica, polyvinyl alcohol, titanium dioxide, micronized silica, fumed silica, glycerol monostearate, magnesium trisilicate, and magnesium stearate, or a mixture thereof. The sealant or barrier layer can be applied from solution (e.g., aqueous) or suspension using any known means, such as a fluidized bed coater (e.g., Wurster coating) or pan coating system. Suitable sealants or barriers include, for example, OPADRY® WHITE Y-1-7000® and OPADRY® OY/B/28920 WHITE®, each of which is available from Colorcon Limited, England.

The present invention also provides an oral dosage form comprising a multiparticulate acarbose as hereinabove defined, in the form of caplets, capsules, particles for suspension prior to dosing, sachets, or tablets. The dosage form can be of any shape suitable for oral administration of a drug, such as spheroidal, cube-shaped oval, or ellipsoidal. The dosage forms can be prepared from the multiparticulates in a manner known in the art and include additional pharmaceutically acceptable excipients, as desired.

Tablets can be formed by any suitable process, examples of which are known to those of ordinary skill in the art. For example, the ingredients can be dry-granulated or wet-granulated by mixing in a suitable apparatus before tabletting. Granules of the ingredients to be tabletted can also be prepared using suitable spray/fluidization or extrusion/spheronization techniques.

Tablets can be designed to have an appropriate hardness and friability to facilitate manufacture on an industrial scale using equipment to produce tablets at high speed. Also, the tablets can be packed or filled in any kind of container. It should be noted that the hardness of tablets, amongst other properties, can be influenced by the shape of the tablets. Different shapes of tablets can be used according to the present disclosure. Tablets can be circular, oblate, oblong, or any other shape. The shape of the tablets can also influence the disintegration rate.

Any of the inventive formulations can be encapsulated in soft or hard gelatin capsules, which can also include any of the excipients described above. For example, the encapsulated dosage form can include fillers, such as lactose and microcrystalline; glidants, such as colloidal silicon dioxide and talc; lubricants, such as magnesium stearate; and disintegrating agents, such as starch (e.g., maize starch). Using capsule filling equipment, the ingredients to be encapsulated can be milled together, sieved, mixed, packed together, and then delivered into a capsule. Lubricants can be present in an amount ranging from about 0.5% (w/w) to about 2.0% (w/w).

All of the embodiments described above, including but not limited to, matrix-based, osmotic pump-based, soft gelatin capsules, and/or membrane-controlled forms, which can further take the form of monolithic and/or multi-unit dosage forms, can have a functional coating. Such coatings generally serve the purpose of delaying the release of the drug for a predetermined period. For example, such coatings can allow the dosage form to pass through the stomach without being subjected to stomach acid or digestive juices. Thus, such coatings can dissolve or erode upon reaching a desired point in the gastrointestinal tract, such as the small intestine.

Such functional coatings can exhibit pH-dependent or pH-independent solubility profiles. Those with pH-independent profiles generally erode or dissolve away after a predetermined period, and the period can be related to the thickness and composition of the coating. Those with pH-dependent profiles, on the other hand, can maintain their integrity while in the acid pH of the stomach, but quickly erode or dissolve upon entering the more basic areas of the gastrointestinal tract.

Thus, a matrix-based osmotic pump-based, or membrane-controlled formulation can be further coated with a functional coating that delays the release of the drug. For example, a membrane-controlled formulation can be coated with an enteric coating that delays the exposure of the membrane-controlled formulation until the small intestine is reached. Upon leaving the acidic stomach and entering the more basic intestine, the enteric coating dissolves. The membrane-controlled formulation then is exposed to gastrointestinal fluid, and then releases the acarbose over an extended period, in accordance with the present disclosure. Examples of functional coatings such as these are well known to those in the art.

In certain embodiments, the acarbose formulations initially delay release of the drug. Following the delay, the formulation rapidly releases the drug.

Additional Pharmaceutically Active Compound

The present invention overcomes the deficiencies and problems in the prior art by providing new and effective methods and formulations for reducing, preventing, and/or managing chronic constipation and constipation as a symptom associated with other diseases and/or conditions. The methods for reducing, preventing, and/or managing chronic constipation involve administering a therapeutically effective amount of acarbose, or a pharmaceutically acceptable salt thereof, to a subject in need of such reduction, prevention, and/or management. Chronic constipation can be associated with at least one bowel condition. Thus, the present invention can also be used to directly or indirectly reduce, prevent, and/or manage such conditions, e.g., lifestyle habits, i.e., low dietary fiber intake, diseases of the peripheral and central nervous system, nonneurological conditions, and functional or idiopathic constipation by the use of acarbose. Examples of conditions with constipation as a symptom thereof that can treat, prevent, and/or manage this symptom according to the present disclosure include, but are not limited to, irritable bowel syndrome (IBS), endocrine disorders, metabolic disturbances, myotonic dystrophy, psychiatric disorders, divertoculosis, hypothyroidism, and other conditions exhibiting constipation as a symptom thereof. Those of ordinary skill in the art will be familiar with other types of gastrointestinal and/or bowel conditions with constipation as a symptom thereof, which can benefit from the present invention.

In some embodiments, the present invention also provides methods and formulations for treating chronic constipation, comprising administering to a subject in need of such treatment a therapeutically effective amount of acarbose, or a pharmaceutically acceptable salt thereof, at least one pharmaceutically acceptable ingredient to control the release of acarbose, in combination with at least one additional pharmaceutically active compound. Combinations can be administered such that acarbose or a pharmaceutically acceptable salt thereof, at least one pharmaceutically acceptable ingredient, and at least one additional pharmaceutically active compound are contained in the same dosage form. Alternatively, the combination can be administered such that acarbose and the at least one additional pharmaceutically active compound are contained in separate dosage forms and are administered concomitantly or sequentially.

Dosages

The acarbose used in accordance with the present invention can be obtained by any method. For example, U.S. Pat. No. 4,904,769 describes such methods, which are incorporated herein by reference for this purpose. Modifications of the protocols described therein and as well as other routes of synthesis, are well known to those of ordinary skill in the art and can be employed in accordance with the present invention.

In accordance with the present invention, the acarbose, or a pharmaceutically acceptable salt thereof, is formulated and/or dosed in a manner that maximizes its therapeutic effects, while minimizing at least one systemic side effect.

The amount of the dose administered, as well as the dose frequency, will vary depending on the particular dosage form used and the route of administration. The amount and frequency of administration will also vary according to the age, body weight, and response of the individual subject. A competent physician without undue experimentation can readily determine typical dosing regimens. It is also noted that the clinician or treating physician will know how and when to interrupt, adjust, or terminate therapy in conjunction with individual subject response.

In general, the total daily dosage for reducing, preventing, and/or managing chronic constipation, with any of the formulations according to the present invention, is from about 5 mg to about 200 mg, or from about 10 mg to about 150 mg, or from about 25 mg to about 100 mg. A single oral dose can be formulated to comprise about 5, 10, 25 mg, 50 mg, 100, 150, 200 mg, or any amount in between.

The pharmaceutical formulations comprising acarbose, or a pharmaceutically acceptable salt thereof, can be administered in single or divided doses, 1, 2, 3, 4, 5, or more times each day. Alternatively, the dose can be delivered at least one time every 2, 3, 4, 5, 6, 7, or more days. In some embodiments, the pharmaceutical formulations are administered once per day.

Release Profiles

Some embodiments of the invention are directed to methods and formulations that employ a formulation having a delayed-release, extended-release and/or mixtures thereof profile.

Optimization of the acarbose release profile can permit one to delay release of the acarbose in a manner such that release can occur at desired gastrointestinal sites, e.g., the small intestine.

Other than in the Examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that can vary depending upon the desired properties to be obtained by the invention. At the very least, and not as an attempt to limit the application of the doctrine of equivalents to the scope of the claims, each numerical parameter should be construed in light of the number of significant digits and ordinary rounding approaches.

Notwithstanding that numerical ranges and parameters setting forth the broad scope of the invention are approximations, the numerical values set forth in the specific examples are reported as precisely as is conventional in the art. Any numerical value, however, inherently contains certain errors necessarily resulting from the standard deviation found in their respective testing measurements.

The present invention is further illustrated by reference to the following examples. It will be apparent to those skilled in the art that many modifications, both to the materials and methods, can be practiced without departing from the purpose and scope of the disclosure.

### EXAMPLES

### Example 1

### Uncoated Instant-Release Acarbose Tablet Formulation

| | | Formulation | | |
|---|---|---|---|---|
| | | A | B | B |
| Ingredient | Function | mg/tab | mg/tab | mg/tab |
| Acarbose | Active | 25.0 | 25.0 | 25.0 |
| Lactose | Diluent | 114.0 | 64.0 | 48.0 |
| Sodium Starch | Disintegrant | 80.0 | 70.0 | 66.0 |
| Glycollate | | | | |
| Avicel PH101 | Binder | 114.0 | 64.0 | 48.0 |
| | Diluent | | | |
| Colloidal Silicone | Glidant | 2.0 | 2.0 | 2.0 |
| Dioxide | | | | |
| Magnesium | Lubricant | 20.0 | 20.0 | 20.0 |
| Stearate | | | | |
| Povidone (PVP, | Binder | 50.0 | 50. | 50.0 |
| polyvinylpyrollidone) | | | | |
| Isopropyl Alcohol* | Solvent | N/A | N/A | N/A |
| Total (mg) | N/A | 385.0 | 295.0 | 259.0 |

*Removed during processing.

Manufacturing Process

Weigh the ingredients using a suitable balance.

Place the acarbose, 50% of the Avicel, and 50% of the lactose in a suitable mixer.

Mix for about 15 minutes until homogenous.

Continue mixing and add the granulating fluid (IPA(Isopropyl alcohol)/PVP solution.

Mix until a suitable granulation end point is achieved. More isopropyl alcohol (IPA) can be added to produce a suitable granule.

Dry the granules until an acceptable level of moisture, e.g., less than 1.0% and IPA, e.g., less than 0.5%, is achieved.

Pass the dry granulate through suitable comminution equipment fitted with a suitably sized screen, e.g., 100-500 micron.

Place the granulate in a blender and add the colloidal silicon dioxide (glidant), the sodium starch glycolate (disintegrant), and the remaining lactose (diluent) and Avicel (binder diluent).

Mix for about 15 minutes.

Add the magnesium stearate (lubricant) and mix for an additional 5 minutes.

Compress the formulation into oval shaped tablets using a suitable tablet machine.

Alternatively, the acarbose is dissolved in IPA (or an alternative solvent) and the PVP is mixed into the dry blend prior to granulation.

### Example 2

### Modified-Release Acarbose Formulations

The instant release tablet formulations (A, B, and/or C) of Example 1 can be coated with a functional coat. Examples of two types of coatings are given below:

Coating One

| Ingredient | Function | Qty % (w/w) | Batch 1 mg/tab |
|---|---|---|---|
| Eudragit L 100 | Polymer | 6.39 | 6.00 |
| Acetyl Tributyl | Plasticizer | 1.60 | 1.50 |
| Citrate | | | |
| Water* | Solvent | 3.26 | N/A |
| Ethanol* | Solvent | 88.75 | N/A |
| Total | N/A | 100.0 | N/A |

*Removed during processing.

Manufacturing Process

Load the tablets into a suitable coating machine.

Spray the polymer coating on to the tablets.

Once the required amount of polymer coating solution has been applied, dry the tablets in the coating machine.

Coating Two

| Ingredient | Weight (g) |
|---|---|
| Eudragit S 12.5 | 5,000 |
| Dibutyl Sebecate | 125 |
| Talc | 312.5 |
| Purified Water* | 300 |
| Isopropyl Alcohol* | 4262.5 |
| Total | 10,000 |

*Removed during processing.

Manufacturing Process

Add the purified water to the isopropyl alcohol and mix for about 10 minutes.

Add the dibutyl sebecate and stir for about 10 minutes.

Add the talc and continue to mix for about 15 minutes.

Finally, add the Eudragit S and mix until homogeneous, e.g., about 30 minutes.

Spray directly onto the instant release tablets using fluidized coating equipment and the method described above.

### Example 3

### In-Vitro Release Test Results

The modified-release tablets of Example 2 based on coating 1 exhibit the following dissolution profile when tested in a USP type I or II apparatus at 50-100 rpm in 900 ml of medium fluid at 37° C.:

after 2 hours in medium 0.01N HCl <10% of drug is released;

subsequently after 1 hour in medium pH 6.8 >50% of drug is release; and

subsequently after 2 hour in medium pH 6.8 >75% of drug released.

The delayed-release tablets of Example 2 based on coating 2 above exhibit a dissolution profile when tested in a USP type I or II apparatus at 50-100 rpm in 900 ml of medium fluid at 37° C.:

after 2 hours in medium 0.01N HCl <10% of drug is released;

subsequently after 1 hour in medium pH 6.8 >10% of drug is released;
2 hours in medium pH 6.8 >20% of drug is released;
4 hours in medium pH 6.8 >40% of drug is released; and
8 hours in medium pH 6.8 >75% of drug is released.

### Example 4

### Modified-Release Acarbose Tablet Formulations

Uncoated Modified-Release Formulations of Metformin Using Methocel Premium at Various Levels. (Wet granulation method).

Matrix Tablet Formulations

The uncoated matrix tablet formulations and processing details are given below:

| | | Formulation | | |
|---|---|---|---|---|
| | | D | E | F |
| Ingredient | Function | mg/tab | mg/tab | mg/tab |
| Acarbose | Active | 25.0 | 25.0 | 25.0 |
| Lactose | Diluent | 114.0 | 64.0 | 48.0 |
| Avicel | Binder | 124.0 | 74.0 | 58.0 |
| PH101 | Diluent | | | |
| Methocel | Controlled | 200.0 | 300.0 | 400.0 |
| Premium | Release | | | |
| CR** | Polymer | | | |
| Colloidal | Glidant | 2.0 | 2.0 | 2.0 |
| Silicon | | | | |
| Dioxide | | | | |
| Magnesium | Lubricant | 10.0 | 10.0 | 10.0 |
| Stearate | | | | |
| PVP | Binder | 50.0 | 50.0 | 50.0 |
| Isopropyl | Solvent | N/A | N/A | N/A |
| Alcohol* | | | | |
| Total (mg) | N/A | 1000 | 1000 | 1068 |

*Removed during processing.
**Methocel grade can be changed or alternatively, a suitable controlled-release polymer can be used.

Weigh the ingredients using a suitable balance.

Place the acarbose, 50% of the Avicel, and 50% of the lactose in a suitable mixer.

Mix for about 15 minutes until homogenous.

Continue mixing and add the granulating fluid (IPA/PVP Solution).

Mix until a suitable granulation end point is achieved. More IPA can be added to produce a suitable granule.

Dry the granules until an acceptable level of moisture, e.g., less than 1.0% and IPA, e.g., less than 0.5%, is achieved.

Pass the dry granulate through suitable comminution equipment fitted with a suitably sized screen, e.g., 100-500 micron.

Place the granulate in a blender and add the colloidal silicon dioxide (glidant), and the remaining lactose (diluent) and Avicel (binder diluent).

Mix for about 15 minutes.

Add the magnesium stearate (lubricant) and mix for an additional 5 minutes.

Compress the formulation into oval shaped tablets (target weight about 1000 mg) using a suitable tablet machine.

Alternatively, the metformin is dissolved in IPA (or an alternative solvent) and the PVP is mixed into the dry blend prior to granulation.

### Example 5

### In Vitro Test Results

The above modified-release tablet formulations (D, E, and F) can be coated with a delayed-release functional coating as described in Example 2.

The modified-release tablets of Example 4 based on coating 1 exhibit a dissolution profile when tested in a USP type I or II apparatus at 50-100 rpm in 900 ml of medium fluid at 37° C.:

after 2 hours in medium 0.01N HCl <10% of drug is released;

subsequently after 1 hour in medium pH 6.8 >20% of drug is released;
2 hours in medium pH 6.8 >30% of drug is released;
4 hours in medium pH 6.8 >50% of drug is released; and
8 hours in medium pH 6.8 >75% of drug is released.

The modified release tablets of Example 4 based on coating 2 exhibit a dissolution profile when tested in a USP type I or II apparatus at 50-100 rpm in 900 ml of medium fluid at 37° C.:

after 2 hours in medium 0.01N HCl <10% of drug is released;

subsequently after 1 hour in medium pH 6.8 >10% of drug is released;
2 hours in medium pH 6.8 >20% of drug is released;
4 hours in medium pH 6.8 >30% of drug is released;
6 hours in medium pH 6.8 >40% of drug is released; and
8 hours in medium pH 6.8 >60% of drug is released.

### Example 6

### Pharmacokinetic Study

A single-dose, five-way crossover study in fifteen healthy volunteers fasting overnight and four hours after dosing is designed to compare and assess the relative bioavailability (the bioavailability obtained by comparing the AUCs when like or unlike dosage forms of the same drug are administered by the same or different routes) of four formulations of acarbose with a commercial reference product (PRECOSE®). The formulations are:

(a) PRECOSE® 25 mg

(b) Delayed Onset 500 mg (A) 25 mg

(c) Delayed Onset Modified Release (D) 25 mg

(d) Delayed Onset Modified Release (E) 25 mg

(e) Delayed Onset Modified Release (F) 25 mg

The fifteen healthy volunteers are dosed on one of the 5 study periods in a randomized crossover manner. Venous blood samples are obtained at regular intervals immediately prior to and following each dosing for a period of up to 48 hours. Plasma concentrations of metformin are measured using standard methods. Individual plasma concentration curves are constructed and individual, mean, and relative pharmacokinetic parameters are estimated including Tₘₐₓ (time at the maximum concentration), Cₘₐₓ (maximum observed concentration), and AUC (area under the plasma concentration versus time curve). The following results are obtained:

AUC and Cₘₐₓ of (b)<80% of (a) AUC and Cₘₐₓ

AUC and Cₘₐₓ of (c)<80% of (a) AUC and Cₘₐₓ

AUC and Cₘₐₓ of (d)<70% of (a) AUC and Cₘₐₓ

AUC and Cₘₐₓ of (e)<60% of (a) AUC and Cₘₐₓ

### Example 7

### Clinical Study

A randomized, dose escalation, placebo controlled study is designed to assess the efficacy of the administered formulation in 60 to 120 patients with functional constipation, defined using the Rome II criteria (modified), i.e., at least three weeks in the previous 3 months of two or more of the following symptoms:
i. Straining in >25% of defecations;
ii. Lumpy or hard stools in >25% of defecations;
iii. Sensation of incomplete evacuation in >25% of defecations;
iv. Sensation of anorectal obstruction/blockage in >25% of defecations;
v. Manual maneuvers to facilitate >25%, of defecations (e.g. digital evacuation, support of pelvic floor); and/or
vi. <3 evacuations per week.
   In addition, loose stools are not present, and there are insufficient criteria for a diagnosis of IBS. Moreover, these patients have no evidence of medical disorders that may cause constipation. Patients are symptomatic on entry in the randomization phase of the study, i.e., in the 8-14 day run-in period, on at least 8 days, which need not be consecutive, patients have lumpy or hard stools in >25% of defecations.

Patients are randomized to one of three groups:

a) Modified Release Acarbose (A);

b) Modified Release Acarbose (E); and

c) Placebo.

Patients randomized to receive treatments a) or b) will receive 25 mg once daily for the first four weeks, 50 mg once daily for the following four weeks and 100 mg once daily for a further four weeks, providing the previous dose was well tolerated. Patients randomized to receive placebo ill receive placebo for the duration of the study.

The primary efficacy endpoint is based on the patient's global impression. Patients receiving metformin answer ‘yes’ to the following question: “do you feel better now after treatment” at least 50% of the time, based on daily diaries, during the dose escalation phase of the study.

Secondary efficacy endpoints include the change from baseline compared to placebo in straining during defecations, stool consistency (Bristol Stool Scale), completeness of evacuation, sensation of anorectal obstruction/blockage, use of manual maneuvers to facilitate defecation, frequency of evacuations, and use of rescue medication, i.e., laxatives.

What is claimed is:

1. A method for treating chronic constipation in a subject in need of such treatment comprising administering to the subject a dosage formulation comprising a therapeutically effective amount of acarbose, or pharmaceutically acceptable salt thereof, and at least one pharmaceutically acceptable ingredient to control the release of the acarbose, wherein following administration, the dosage formulation releases the acarbose distal to the gastrointestinal sites at which acarbose is absorbed.

2. The method according to claim 1, wherein the chronic constipation is a symptom of irritable bowel syndrome.

3. The method according to claim 1, wherein the acarbose is administered to the subject orally.

4. The method according to claim 1, wherein the dosage formulation is chosen from delayed-release, extended-release, and mixtures thereof.

5. The method according to claim 1, wherein the dosage formulation further comprises at least one additional pharmaceutically active agent.

6. The method according to claim 5, wherein the at least one additional pharmaceutically active compound is capable of relieving constipation.

7. The method according to claim 5, wherein the at least one additional pharmaceutically active compound is metformin.

8. The method according to claim 7, wherein the metformin is in a form chosen from immediate release and modified release.

9. The method according to claim 1, wherein the dosage formulation is in a tablet form.

10. The method according to claim 9, wherein the dosage formulation is in a hydrophilic matrix tablet form.

11. The method according to claim 1, wherein the dosage formulation provides a daily dose ranging from 5 mg to 200 mg.

12. The method according to claim 11, wherein the daily dose is chosen from single and divided doses.

13. The method according to claim 1, wherein the constipation is treated, while minimizing at least one side effect associated with the administration of a conventional formulation of acarbose, or a pharmaceutically acceptable salt thereof.

14. The method according to claim 1, wherein the dosage formulation releases the acarbose distal to the duodenum of the gastrointestinal tract.

15. The method according to claim 1, wherein the dosage formulation releases the acarbose distal to the jejunum of the gastrointestinal tract.

16. The method according to claim 1, wherein the dosage formulation releases the acarbose distal to the ileum of the gastrointestinal tract.

17. The method according to claim 1, wherein the dosage formulation releases the acarbose after passing through the stomach of the subject.
